(19) United States
(12) Patent Application Publication
(10) Pub. No.: US 2012/0190751 A1
(43) Pub. Date: Jul. 26, 2012

(54) COMPOUNDS FOR TREATMENTS OF INFLAMMATION

(75) Inventors: Niklas Palmqvist, Malmo (SE); Anders Sjödin, Marieholm (SE); Christina Wenglén, Bjarred (SE); Christofer Flood, Lerum (SE); Lennart Lundberg, Billdal (SE); Elisabeth Seifert, Uppsala (SE); Per Lek, Uppsala (SE); Arne Boman, Uppsala (SE)

(73) Assignee: ANA-MAR AB, Goteborg (SE)

(21) Appl. No.: 13/388,174

(22) PCT Filed: Jul. 30, 2010
(86) PCT No.: PCT/GB2010/001457

§ 371 Date: Apr. 13, 2012

(30) Foreign Application Priority Data
Jul. 31, 2009 (GB) 0913427.1
Mar. 31, 2010 (GB) 1005495.5
Jun. 24, 2010 (GB) 1010671.4

(51) Int. Cl. A61K 31/155; A61P 19/02; A61P 25/00; A61P 29/00

(52) U.S. Cl. 514/632

(57) ABSTRACT

The present invention relates to the use of benzylideneaminoguanidines for the treatment of inflammation and pain. In one preferred embodiment, the invention relates to the use of N-(2-chloro-3,4-dimethoxybenzylideneamino)guanidine for the treatment of rheumatoid arthritis.

The present invention relates to the use of certain benzylideneaminoguanidines for the treatment of inflammation and pain.

Rheumatoid arthritis (RA) is a chronic, inflammatory autoimmune disease that causes the immune system to attack the joints. It results in a disabling and painful condition that can lead to substantial loss of mobility due to joint destruction and the associated pain. The aetiology behind RA is largely unknown. RA affects about 1% of adults; it is two to three times more prevalent in women than in men. RA may begin as early as infancy, but onset usually occurs in the fifth or sixth decade.

Various treatments have been tried. Non-pharmacological treatments include physical therapy and occupational therapy. Analgesics (painkillers) and anti-inflammatory drugs, as well as steroids, have been used to suppress the symptoms, while disease-modifying anti-rheumatic drugs (DMARDs) are often required to inhibit or halt the underlying immune process and prevent long-term damage.

There remains a need, however, for alternative pharmaceutical treatments of inflammation and pain.

In inflammatory conditions such as RA, serotonin (5-Hydroxytryptamine, 5-HT) and its receptors play important roles.

The serotonin system, with its many receptors, is involved in many signalling events in the body. (For a review, see Berger et al. Annu. Rev. Med. (2009), 60:355-66). For example, serotonin regulates biological processes such as cardiovascular function, bowel motility and bladder control. A greater understanding of serotonin function has emerged during the last two decades with the cloning of at least 15 serotonin receptors; these are grouped into seven families based on signalling mechanisms.

Other important advances have included the subsequent development of receptor-specific knockout mice, and the development of receptor subtype-selective drugs. These advances have shown that serotonin has critically important functions in many human organ systems, including inflammatory processes and pain.

In inflammatory conditions such as rheumatoid arthritis, platelets take up serotonin from the plasma via the serotonin transporter, and serotonin is then secreted by the platelets during activation at the inflammatory site. This released serotonin induces production of pro-inflammatory cytokines by binding to inflammatory cells, e.g. macrophages, T-cells and fibroblasts. For example, several studies have shown a correlation between the level of 5-HT content, plasma levels and disease activity in arthritis patients.

Several receptors such as 5-HT, 5-HT_{2A}, 5-HT_{2C}, 5-HT₄, 5-HT₇ have been linked to 5-HT's effects in inflammation. However, it is notable that the 5-HT_{2B} receptor has never previously been directly linked to inflammation.

The 5-HT_{2B} receptor has been linked to pulmonary artery hypertension (PAH) and antagonists may be useful in treating PAH. Agonists to 5-HT_{2B} receptor have been described (for example fen/phe story—valvular heart disease and obesity). Involvement of the 5-HT_{2B} receptor in cardiac hypertrophy and a link to regulation of interleukin-6, interleukin-1beta, and TNF-alpha cytokine production has been published. The 5-HT_{2B} receptor has also been discussed in indications such as constipation and migraine.

It has now surprisingly been found that 5-HT_{2B} receptor is directly and closely related to inflammatory processes peripherally. In particular, it has been found that this receptor sub-type is expressed in pannus as well as in macrophages. The presence of 5-HT_{2B} receptors on the invasive and aggressive synovial cells in the pannus tissue, i.e. the synovial fibroblasts and macrophages, makes them important as targets for modulating the inflammatory response in RA. By antagonizing these receptors, the expression of IL-6 and TNF-alpha is decreased, an effect which is most relevant when treating arthritis.

It is an object therefore to provide compounds and certain benzylideneaminoguanidines in particular or pharmaceutically acceptable salts thereof which are capable of binding to 5-HT_{2B} receptors and which are capable of use for the treatment of inflammation, pain and other disorders which are associated with 5-HT_{2B} receptors.

Various benzylideneaminoguanidines and hydroxyguanidines have previously been described as melanocortin receptor ligands. In particular, WO02/11715 discloses 164 benzylideneaminoguanidines and hydroxyguanidines as being melanocortin receptor ligands. The compounds disclosed therein are said to be useful for the treatment of a wide range of disorders which are associated with the melanocortin receptors, including mental disorders, dysfunctions of the endocrine and hormonal systems, sexual dysfunction, inflammation, drug-induced disorders of the blood and lymphoid system, fast allergic disorders, cardiovascular disorders, pain, stimulation of pigment formation, stimulation of second messenger elements and for tagging with a toxic agent. The Examples of WO02/11715 are said to illustrate the potency of the compounds disclosed therein for the treatment of mental disorders. More specifically, data on the binding of N-(3-bromo-4-methoxybenzylideneamino)-N′-hydroxyguanidine and N-(5-chloro-2-nitrobenzylideneamino)-N′-hydroxyguanidine to MC1, MC3, MC4 and MC5 receptors is disclosed. This document does not, however, specifically disclose the use of the benzylideneaminoguanidines referred to herein for the treatment of inflammation or pain.

In one aspect, the invention provides a pharmaceutical composition comprising a compound of formula (I)

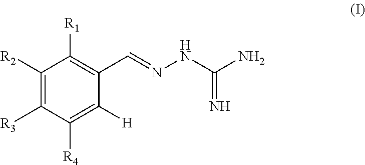

wherein

R1 is Cl, MeO or H

R2 is Cl, MeO or H

R3 is Cl or MeO

R4 is Cl, H or NO₂

with the proviso at least one of R1-R3 must be Cl

and at least one of R1-R3 must be MeO, or a pharmaceutically acceptable salt thereof, for the treatment of inflammation or pain.

In some preferred compounds, R1 is Cl. In other preferred compounds, R2 is MeO. In other preferred compounds, R3 is MeO. In yet other preferred compounds, R4 is H.

In some embodiments of the invention, there is provided a pharmaceutical composition comprising a compound of formula II:

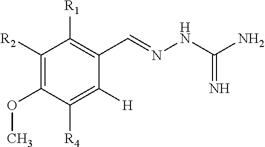

wherein

R1 is Cl or MeO, preferably Cl,

R2 is Cl or MeO, preferably MeO,

R4 is Cl or H, preferably H,

with the proviso that at least one of R1 and R2 must be Cl,

or a pharmaceutically acceptable salt thereof, for the treatment of inflammation or pain.

Most preferably, the compound of formula I or II is N-(2-chloro-3,4-dimethoxybenzylideneamino)guanidine.

Compounds of formula (I) or (II) and/or their pharmaceutically acceptable salts have valuable pharmacological properties, making them useful for the treatment of inflammation such as inflammation regulated by the 5-HT system, inflammation related to the production of nitric oxide, inflammation related to increased amounts (upregulated amounts) of inducible nitric oxide synthase, inflammation related to activation of transcriptional activators, inflammation related to nuclear factor kappa beta, inflammation related to macrophages, neutrophils, monocytes, keratinocytes, fibroblasts, melanocytes, pigment cells and endothelial cells, inflammation related to increased production and/or release of inflammatory cytokines, such as e.g. interleukins, in particular interleukin 1 (IL-1), interleukin 6 (IL-6) and tumour necrosis factor α (TNF-α).

In the present specification, “increased production” refers to increased formation, increased release, or increased amount of an endogenous compound locally, regionally or systemically in a patient compared to the amount of said endogenous compound in a healthy individual. In the present specification, “upregulated” refers to an increased activity or amount of the compound compared with that in a healthy individual.

In the present specification, “decreased production” refers to decreased formation, decreased release, or decreased amount of an endogenous compound in a patient compared to the amount of said endogenous compound in a healthy individual. In the present specification, “downregulated” refers to a decreased activity or amount of the compound compared with that in a healthy individual.

In particular, positive treatment effects or preventive effects may be seen in conditions where inflammation or an inflammatory-like condition is caused by or being associated with one or more of the following: allergy, hypersensitivity, bacterial infection, viral infection, inflammation caused by toxic agent, fever, autoimmune disease, radiation damage by any source including UV-radiation, X-ray radiation, γ-radiation, α- or β-particles, sun burns, elevated temperature or mechanical injury. Moreover, inflammation due to hypoxia, which is optionally followed by reoxygenation of the hypoxic area, is typically followed by severe inflammation, which condition may be positively affected by treatment with a compound of the invention.

In very specific embodiments of the invention, a compound of the invention may be administered for the prevention or therapeutic treatment of inflammatory diseases of the skin (including the dermis and epidermis) of any origin, including skin diseases having an inflammatory component. Specific examples of this embodiment of the invention include treatment of contact dermatitis of the skin, sunburns of the skin, burns of any cause, and inflammation of the skin caused by chemical agents, psoriasis, vasculitis, pyoderma gangrenosum, discoid lupus erythematosus, eczema, pustulosis palmo-plantaris, and phemphigus vulgaris.

Also comprised by the invention is a compound of formula (I) or (II) or a pharmacologically acceptable salt thereof for the treatment of an inflammatory disease in the abdomen, including an abdominal disease having an inflammatory component. Specific examples of the treatment of such a disease with a compound of the invention are gastritis, including one of unknown origin, gastritis pemiciosa (atrophic gastritis), ulcerous colitis (colitis ulcerosa), morbus Crohn, systemic sclerosis, ulcus duodeni, coeliac disease, oesophagitis and ulcus ventriculi.

Comprised by the invention is also a compound of formula (I) or (II) or a pharmacologically acceptable salt thereof for the treatment of systemic or general and/or local immunological diseases, including those of an autoimmune nature, and other inflammatory diseases of a general nature. Specific examples include treatment of rheumatoid arthritis, psoriatic arthritis, systemic sclerosis, polymyalgia rheumatica, Wegener's granulomatosis, sarcoidosis, eosinophilic fasceitis, reactive arthritis, Bechterew's disease, systemic lupus erythematosus, arteritis temporalis, Behcet's disease, morbus Burger, Good Pastures' syndrome, eosinophilic granuloma, fibromyalgia, myositis, and mixed connective tissue disease. Included therein is also arthritis, including arthritis of unknown origin.

Further included in the invention is a compound of formula (I) or (II) or a pharmacologically acceptable salt thereof for the treatment of a disease of the peripheral and/or central nervous system related to inflammation. Included in this aspect of the invention is the treatment of cerebral vasculitis, multiple sclerosis, autoimmune ophthalmitis and polyneuropathia. Comprised by the invention is also the administration of a compound of the invention for the treatment of an inflammation of the central nervous system to prevent apoptotic cell death. Moreover, as some of the compounds of the invention show a distinct ability to induce nerve regeneration, positive treatment effects are often seen in central nervous system diseases involving damage of cells in this region. This aspect of the invention also includes treatment of traumatic injuries to the central nervous system, brain edema, multiple sclerosis, Alzheimer's disease, bacterial and viral infections in the central nervous system, stroke, and haemorrhagia in the central nervous system.

Comprised by the invention is also a compound of formula (I) or (II) or a pharmacologically acceptable salt thereof for the treatment of diseases of the eye and tear glands related to inflammation. Specific examples of such diseases comprise anterior and posterior uveitis, retinal vasculitis, optic neuritis, optic neuromyelitis, Wegener's granulomatosis, Sjögren's syndrome, episcleritis, scleritis, sarcoidosis affecting the eye and polychondritis affecting the eye.

Comprised by the invention is also a compound of formula (I) or (II) or a pharmacologically acceptable salt thereof for the treatment of diseases of the ear related to inflammation, specific examples of which include polychondritis affecting the ear and external otitis.

Comprised by the invention is also a compound of formula (I) or (II) or a pharmacologically acceptable salt thereof for the treatment of diseases of the nose related to inflammation, specific examples of which are sarcoidosis, polychondritis and mid-line granuloma of the nose.

Comprised by the invention is also a compound of formula (I) or (II) or a pharmacologically acceptable salt thereof for the treatment of diseases related to inflammation of the mouth, pharynx and salivary glands. Specific examples include Wegener's granulomatosis, mid-line granuloma, Sjögren's syndrome and polychondritis in these areas.

Included in the invention is also a compound of formula (I) or (II) or a pharmacologically acceptable salt thereof for the treatment of diseases related to inflammation in the lung. Specific examples include treatment of idiopathic alveolitis, primary pulmonary hypertension, bronchitis, chronic bronchitis, sarcoidosis, alveolitis in inflammatory systemic disease, pulmonary hypertension in inflammatory systemic disease, Wegener's granulomatosis and Good Pastures' syndrome.

Comprised by the invention is also a compound of formula (I) or (II) or a pharmacologically acceptable salt thereof for the treatment of diseases related to the inflammation of the heart. Specific examples include treatment of pericarditis, idiopathic pericarditis, myocarditis, Takayasus' arteritis, Kawasaki's disease, coronary artery vasculitis, pericarditis in inflammatory systemic disease, myocarditis in inflammatory systemic disease, endocarditis and endocarditis in inflammatory systemic disease.

Comprised by the invention is also a compound of formula (I) or (II) or a pharmacologically acceptable salt thereof for the treatment of diseases related to inflammation of the liver. Specific examples include treatment of hepatitis, chronic active hepatitis, biliary cirrhosis, hepatic damage by toxic agents, interferon induced hepatitis, hepatitis induced by viral infection, liver damage induced by anoxia and liver damage caused by mechanical trauma.

Comprised by the invention is also a compound of formula (I) or (II) or a pharmacologically acceptable salt thereof for the treatment of diseases related to inflammation of the pancreas. Specific examples include treatment (and prevention) of diabetes mellitus, acute pancreatitis and chronic pancreatitis.

Comprised by the invention is also a compound of formula (I) or (II) or a pharmacologically acceptable salt thereof for the treatment of diseases related to the inflammation of the thyroidea. Specific examples of these embodiments of the invention include treatment of thyreoiditis, autoimmune thyreoiditis and Hashimoto's thyreoiditis.

Comprised by the invention is also a compound of formula (I) or (II) or a pharmacologically acceptable salt thereof for the treatment of diseases related to inflammation of the kidney. Specific examples include treatment of glomerulonephritis, glomerulonephritis in systemic lupus erythematosus, periarteritis nodosa, Wegener's granulomatosis, Good-Pastures' syndrome, HLAb27 associated diseases, IgA nephritis (IgA=Immunoglobulin A), pyelonephritis, chronic pyelonephritis and interstitial nephritis.

Comprised by the invention is also a compound of formula (I) or (II) or a pharmacologically acceptable salt thereof for the treatment of diseases related to the inflammation of the joints. Specific examples include treatment of Bechterew's disease, psoriatic arthritis, rheumatoid arthritis, arthritis in colitis ulcerosa, arthritis in morbus Crohn, affection of joints in systemic lupus erythematosus, systemic sclerosis, mixed connective tissue disease, reactive arthritis, Reiter's syndrome. Moreover, included in this embodiment of the invention is treatment of arthrosis (osteoarthritis) of any joint, in particular arthrosis of finger joints, the knee and the hip.

Comprised by the invention is also a compound of formula (I) or (II) or a pharmacologically acceptable salt thereof for the treatment of diseases related to the inflammation of blood vessels. Specific examples include treatment of arteritis temporalis, periarteritis nodosa, arteriosclerosis, Takayasus' arteritis and Kawasaki's disease. Particularly advantageous is the capacity of some compounds of the invention to afford protection against and prevention of arteriosclerosis. This is in part due to the capacity of some compounds of formula (I) or the pharmacologically acceptable salts thereof to prevent the induction of inducible nitric oxide synthesis (iNOS) caused by the action of oxidized Low Density Lipoprotein on endothelial cells and blood vessel walls.

Comprised by the invention is also a compound of formula (I) or (II) or a pharmacologically acceptable salt thereof for the treatment of inflammation related to infections of any origin. Specific examples include treatment of inflammation secondary to infection caused by virus, bacteria, helminths and protozoae.

Comprised by the invention is also a compound of formula (I) or (II) or a pharmacologically acceptable salt thereof for the treatment of inflammations related to trauma and/or tissue injury of any origin.

Preferably, the compound of formula (I) or (II) is used for the treatment of rheumatoid arthritis.

The invention particularly relates to N-(2-chloro-3,4-dimethoxybenzylideneamino)guanidine for the treatment of rheumatoid arthritis.

Compounds of formula (I) or (II) or the pharmaceutically acceptable salts thereof have valuable pharmacological properties, making them useful for the treatment of pain such as pain of central origin, pain seen after damage to the CNS, stroke, infarction, pain of peripheral origin, chronic pain, neuropathies and disorders where a treatment effect is achieved by stimulation of receptors in the periaqueductal grey area.

Preferably the pain is pain associated with inflammatory conditions.

In other embodiments, the pain is preferably associated with inflammation in the joints or pain associated with RA.

Examples of pharmaceutically acceptable salts of the compounds of the present invention, include acid addition salts with inorganic acids (e.g., hydrochloric acid, hydrobromic acid, hydroiodic acid, sulphuric acid, nitric acid, phosphoric acid and the like), organic acids (e.g., formic acid, acetic acid, propionic acid, oxalic acid, malonic acid, succinic acid, fumaric acid, maleic acid, lactic acid, malic acid, tartaric acid, citric acid, methanesulphonic acid, ethanesulphonic acid, aspartic acid, glutamic acid and the like). Depending on the substituent, the compound of the invention might form a salt with a base, for example, salts with inorganic bases containing metals such as sodium, potassium, magnesium, calcium, aluminium and the like or organic bases (e.g., methylamine, ethylamine, ethanolamine, lysine, ornithine and the like), ammonium salt.

In addition, the present invention also includes various hydrates, solvates and polymorphic substances of the compound (I) and (II) and salts thereof.

Pharmaceutical compositions comprising a compound or compounds of the present invention or a salt thereof as the active ingredient may additionally comprise one or more carriers, fillers and other additive agents which are generally used in the preparation of medicines.

The administration may be by oral administration by tablets, pills, capsules, granules, powders, solutions and the like, or parenteral administration by injections (e.g., intravenous, intramuscular and the like), suppositories, percutaneous preparations, transnasal preparations, inhalations and the like. Preferably, the pharmaceutical composition is formulated for oral use. In some embodiments, oral doses of about 5 mg, 25 mg, 50 mg, 75 mg, 100 mg or 150 mg are administered, preferably to a human subject, and preferably once or twice daily.

The dose is optionally decided in response to each case by taking symptom, age, sex and the like of the subject to be administered into consideration, but in the case of oral administration, it is generally approximately from 0.001 mg/kg to 100 mg/kg per day per adult, and this is administered once or by dividing into 2 to 4 times. In some embodiments, the dose of the oral administration may be 0.05 mg/kg to 5.0 mg/kg per adult.

When intravenously administered, it is generally administered once to two or more times a day within the range of from 0.0001 mg/kg to 10 mg/kg per day per adult. In the case of transnasal administration, it is generally administered once to two or more times a day within the range of from 0.0001 mg/kg to 10 mg/kg per day per adult. In addition, in the case of inhalation, it is generally administered once to two or more times a day within the range of from 0.0001 mg/kg to 1 mg/kg per day per adult.

In a solid composition for oral administration, one or more of the compounds referred to herein may be mixed with at least one inert filler such as lactose, mannitol, glucose, hydroxypropylcellulose, microcrystalline cellulose, starch, polyvinyl pyrrolidone, aluminium magnesium silicate or the like. The composition may also contain inert additives such as lubricants (e.g. magnesium stearate), disintegrators (e.g. carboxymethylstarch sodium), and solubilizing agents, and the like. The tablets or pills may be coated with a sugar-coating or a gastric- or enteric-coating.

With regard to liquid compositions for oral administration, pharmaceutically acceptable emulsions, solutions, suspensions, syrups, elixirs and the like may be included, which may contain an inert solvent such as purified water or ethanol. In addition to the inert solvent, this composition may contain auxiliary agents (e.g. solubilizing agents, moistening agents, suspending agents and the like), sweeteners, correctives, aromatics and/or antiseptics.

With regard to injections for parenteral administration, sterile aqueous or non-aqueous solutions, suspensions and emulsions may be included. As the aqueous solvent, for example, distilled water for injection and physiological saline may be included. Examples of a non-aqueous solvent include propylene glycol, polyethylene glycol, plant oils (e.g. olive oil or the like), alcohols (e.g. ethanol or the like), polysorbate 80, and the like. Such a composition may further contain tonicity agents, antiseptics, moistening agents, emulsifying agents, dispersing agents, stabilizing agents and/or solubilizing agents. These are generally sterilized by, for example, filtration through a bacteria retaining filter, formulation of bactericides or irradiation. In addition, they can also be used by producing a sterile solid compositions and dissolving or suspending them in sterile water or a sterile solvent for injection prior to use.

Inhalations, transmucosal preparations transnasal preparations and the like may be used in a solid, liquid or semisolid form and can be produced in accordance with conventionally known methods. For example, excipients such as lactose, starch or the like, as well as a pH-adjusting agent, an antiseptic, a surfactant, a lubricant, a stabilizer, and/or a thickener and the like, may be optionally added. An appropriate device for inhalation or blowing may be used for the administration. For example, using a conventionally-known device such as a measured administration inhalation device or the like or a sprayer, a compound can be administered alone or as a powder in a prescribed mixture, or as a solution or suspension by a combination with a medicinally-acceptable carrier. The dry-powder inhaler or the like may be for single or multiple administration use, and a dry-powder or a powder-containing capsule may be used. Alternatively, it may be in a form such as a pressurized aerosol spray or the like, which uses suitable gas such as chlorofluoroalkane, hydrofluoroalkane, carbon dioxide or the like.

The application discloses for the first time an association between 5-HT_{2B} receptors and inflammation and pain. Knowledge of this relationship provides methods of obtaining other compounds for use in the treatment of inflammation and/or pain.

The invention therefore also provides a method for identifying test compounds having an anti-inflammatory effect, comprising the steps:

(i) contacting a test compound with a 5-HT_{2B} receptor in vitro, and

(ii) determining the binding capacity of the test compound for the 5-HT_{2B} receptor, wherein a test compound with a K; of less than 1 μM or preferably equal to/less than 0.5 μM is identified as a compound having an anti-inflammatory effect.

The invention also provides a method for identifying test compounds having an anti-inflammatory effect, comprising the steps:
(i) contacting a test compound with a 5-HT_{2B} receptor in vitro,
(ii) determining whether or not the test compound is an antagonist of the 5-HT_{2B} receptor,
   wherein antagonists of the 5-HT_{2B} receptor are compounds having an anti-inflammatory effect.

The invention further provides a method for identifying test compounds having an analgesic effect, comprising the steps:
(i) contacting a test compound with a 5-HT_{2B} receptor in vitro, and
(ii) determining the binding capacity of the test compound for the 5-HT_{2B} receptor,
   wherein a test compound with Kᵢ of less than 1 μM or preferably equal to/less than 0.5 μM is identified as a compound having an analgesic effect.

The invention also provides a method for identifying test compounds having an analgesic effect, comprising the steps:
(i) contacting a test compound with a 5-HT_{2B} receptor in vitro,
(ii) determining whether or not the test compound is an antagonist of the 5-HT_{2B} receptor,
   wherein antagonists of the 5-HT_{2B} receptor are compounds having an analgesic effect.

As used herein, the term “antagonist of the 5-HT_{2B} receptor” is defined as a test compound with an IC₅₀ of less than 100 μM in a tissue functional pharmacology assay, in which 0.1 μM α-methyl serotonin induces contraction in the rat stomach fundus from Wistar rats. (Cohen M L, Fludzinski L A. Contractile serotonergic receptor in rat stomach fundus. J. Pharmacol. Exp. Ther. (1987 October); 243(1):264-269.)

In a further embodiment, the invention provides a pharmaceutical composition comprising a 5-HT2B receptor ligand for the treatment of inflammation. Preferably, the pharmaceutical composition comprises a 5-HT2B receptor ligand, wherein the ligand is a 5-HT2B receptor antagonist. Examples of suitable 5-HT2B receptor antagonists are given in Table 1:

TABLE 1
5HT2B receptor antagonists

| Compound | Synonyms | Affinity | Units |
|---|---|---|---|
| 1-naphthyl- | | 8.4-9.0 | pKᵢ |
| piperazine | | | |
| agomelatine | N-[2-(7-methoxynaphthalen-1-yl)ethyl]acetamide | 6.6 | pKᵢ |
| amesergide | LY237733 | 8.0 | pKᵢ |
| | N-Cyclohexyl-1-isopropyl-6-methylergoline-8- | | |
| | carboxamide | | |
| AMI-193 | 8-[3-(4-fluorophenoxy)propyl]-1-phenyl-1,3,8- | 6.0 | pKᵢ |
| | triazaspiro[4.5]-decan-4-one | | |
| apomorphine | 5,6,6a,7-Tetrahydro-6-methyl-4H-dibenzo[de,g] | 6.9 | pKᵢ |
| | quinolin-10,11-diol | | |
| bromocriptine | 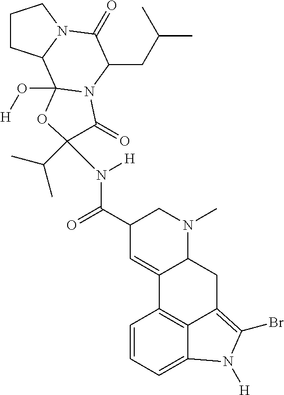 | 7.3 | pKᵢ |
| clozapine | Asaleptin | 8.0-8.8 | pKᵢ |
| | Clorazil | | |
| | Clozapin | | |
| | Clozaril | | |
| | Fazaclo | | |
| | Iprox | | |
| | Leponex | | |
| | Lepotex | | |
| EGIS-7625 | 1-benzyl-4-[(2-nitro-4-methyl-5-amino)-phenyl]- | 9.0 | pKᵢ |
| | piperazine | | |
| F-16615 | 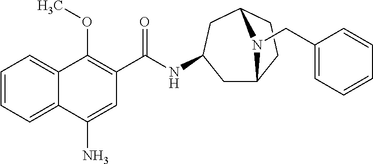 F-16615 Dopamine D2 and 5-HT_{2B} antagonist/5-HT_{1A} receptor partial agonist | | |
| fluoxetine | 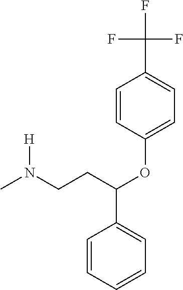 | 5.3 | pKᵢ |
| haloperidol | 4-[4-(4-chlorophenyl)-4-hydroxy-1-piperidyl]-1-(4- | 5.8-6.4 | pKᵢ |
| | fluorophenyl)-butan-1-one | | |
| | Aloperidin | | |
| | Aloperidol | | |
| | Aloperidolo | | |
| | Brotopon | | |
| | Eukystol | | |
| | Galoperidol | | |
| | Haldol | | |
| | Halopoidol | | |
| | Serenelfi | | |
| ketanserin | 3-[2-[4-(4-fluorobenzoyl)-1-piperidyl]ethyl]-1H- | 6.1-6.7 | pKᵢ |
| | quinazoline-2,4-dione | | |
| L-741,626 | 3-[[4-(4-Chlorophenyl)-4-hydroxypiperidin-1-yl]methyl- | 6.2 | pKᵢ |
| | 1H-indole | | |
| lisuride | 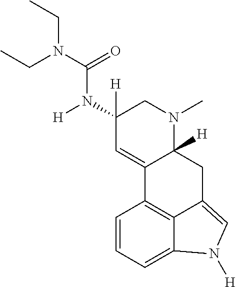 | 8.9 | pKᵢ |
| LY53857 | 6-Methyl-1-(1-methylethyl)ergoline-8β-carboxylic | 8.2 | pKᵢ |
| | acid | | |
| | 2-hydroxy-1-methylpropyl ester maleate salt | | |
| LY86057 | Antagonist | 7.9 | pKᵢ |
| LY272,015 | 1-[(3,4-Dimethoxyphenyl)methy]-2,3,4,9-tetrahydro- | | |
| | 6-methyl-1H-pyrido[3,4-b]indole hydrochloride | | |
| MDL-100,907 | R(+)-α-(2,3-dimethoxyphenyl)-1-[2-(4-fluorophenyl- | 6.0 | pKᵢ |
| | ethyl)]-4-piperidine-methanol | | |
| MDL-11,939 | α-phenyl-1-(2-phenylethyl)-4-piperidinemethanol | 5.5 | pKᵢ |
| melatonin | 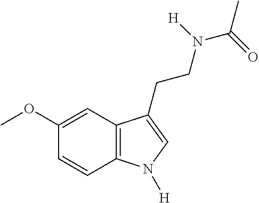 | 5.2 | pKᵢ |
| mesulergine | 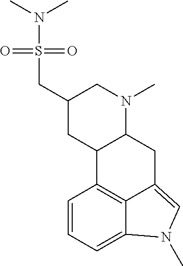 | 7.4 | pKᵢ |
| metergoline | 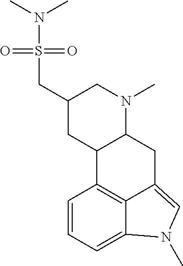 | 8.8 | pKᵢ |
| methiothepin | 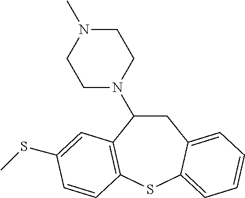 | 8.7-9.2 | pKᵢ |
| mianserin | (±)-2-methyl-1,2,3,4,10,14b- | | |
| | hexahydrodibenzo[c,f]pyrazino[1,2-a]-azepine | 7.9-8.8 | pKᵢ |
| | (+-)-Athymil | | |
| | Mianserina | | |
| | Mianserine | | |
| | Mianserinum | | |
| | Mianseryna | | |
| | (+-)-Norval | | |
| norfluoxetine | 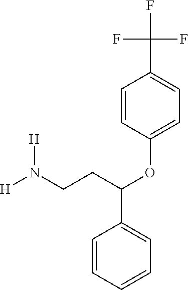 | 5.3 | pKᵢ |
| PGN1091 | | | |
| PGN1164 | | | |
| piboserod | N-((1-Butyl-4-piperidyl)-methyl)-3,4-dihydro-2H- | 6.6 | pKᵢ |
| | (1,3)oxazino(3,2-a)-indole-10-carboxamide | | |
| | SB207256 | | |
| | SB 207256 | | |
| | SB 207266 | | |
| pindolol | 1-(1H-indol-4-yloxy)-3-(propan-2-ylamino)-propan-2-ol | 5.7 | pKᵢ |
| | Betapindol | | |
| | Calvisken | | |
| | Carvisken | | |
| | Decreten | | |
| | Durapindol | | |
| | Pectobloc | | |
| | Pinbetol | | |
| | Prinodolol | | |
| | Visken | | |
| piribedil | 2-[4-(1,3-benzodioxol-5-ylmethyl)piperazin-1-yl] | 5.9 | pKᵢ |
| | pyrimidine | | |
| PRX-08066 | 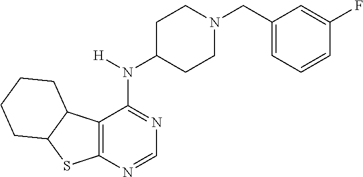 | | |
| rauwolscine | alpha-Yohimbine | 7.4 | pKᵢ |
| | Corynanthidine | | |
| | Isoyohimbine | | |
| | Mesoyohimbine | | |
| ritanserin | 4-[2-[4-[bis(4-fluorophenyl)methylidene]-1- | 8.7-9.2 | pKᵢ |
| | piperidyl]ethyl]-3-methyl-9-thia-2,6-diazabicyclo | | |
| | [4.3.0]nona-1,3,7-trien-5-one | | |
| | Ritanserina | | |
| | Ritanserine | | |
| | Ritanserinum | | |
| | Tiserton | | |
| roxindole | 3-[4-(4-phenyl-3,6-dihydro-2H-pyridin-1-yl)butyl]- | 7.5 | pKᵢ |
| | 1H-indol-5-ol | | |
| RS-102221 | 8-[5-(2,4-dimethoxy-5-(4-trifluoromethylphenyl- | 6.0-6.1 | pKᵢ |
| | sulphonamido)-phenyl-5-oxopentyl]-1,3,8-triazaspiro | | |
| | [4.5]decane-2,4-dione | | |
| RS-127445 | (2-amino-4-(4-fluoronaphth-1-yl)-6- | 9.0 | pKᵢ |
| | isopropylpyrimidine)-ylcarbamoyl]indoline | | |
| RS-127445-190 | | 9.5 | pKᵢ |
| S33084 | (3aR,9bS)-N-[4-(8-cyano-1,3a,4,9b-tetrahydro-3H- | 6.8 | pKᵢ |
| | benzopyrano[3,4-c]pyrrole-2-yl)-butyl]- | | |
| | (4-phenyl)benzamide | | |
| sarpogrelate | 4-[1-dimethylamino-3-[2-[2-(3- | 6.6 | pKᵢ |
| | methoxyphenyl)ethyl]phenoxy]- | | |
| | propan-2-yl]oxy-4-oxo-butanoic acid | | |
| SB 200646 | N-(1-Methyl-1H-indol-5-yl)-N′-3-pyridinylurea | 7.4 | pKᵢ |
| SB 204741 | N-(1-methyl-1H-indo-5yl)-N′-(3-methyl-5- | 6.9 | pKᵢ |
| | isothiazolyl)urea | | |
| SB 206553 | 5-methyl-1-(3-pyridylcarbamoyl)-1,2,3,5- | 7.6-8.5 | pKᵢ |
| | tetrahydrolpyrrolo[2,3-f]indole | | |
| SB 215505 | 6-chloro-5-methyl-1-(5-quinolylcarbamoyl)indoline | 8.3 | pKᵢ |
| SB 221284 | 5-(methylthio)-1-(3-pyridylcarbamoyl-6-trifluoromethyl)indoline | 8.6 | pKᵢ |
| SB 224289 | 1′-methyl-5-[[2′-methyl-4′-(5-methyl-1,2,4- | 5.9 | pKᵢ |
| | oxadiazol-3-yl)biphenyl-4-yl]carbonyl]-2,3,6,7- | | |
| | tetrahydrospiro[furo[2,3-f]indole-3,4′-piperidine | | |
| | hydrochloride | | |
| | 1′-Methyl-5-[[2′-methyl-4′-(5-methyl-1,2,4- | | |
| | oxadiazol-3-yl)biphenyl-4-yl]carbonyl]-2,3,6,7- | | |
| | tetrahydrospiro [furo [2,3-f]indole-3,4'-piperidine] | | |
| | oxalate | | |
| SB 228357 | 1-5[-fluoro-3-(3-pyridyl)phenyl-carbamoyl]-5- | 8.0-8.1 | pKᵢ |
| | methoxy-6-trifuoromethylindoline | | |
| SB 242084 | 6-chloro-5-methyl-1-[6-(-methylpyridin-3-yloxy) | 6.8-7.0 | pKᵢ |
| | pyridin-3-ylcarbomyl]indoline | | |
| SB 243213 | 5-Methyl-1-[[2-[(2-methyl-3-pyridyl)oxy]-5- | 7.0 | pKᵢ |
| | pyridyl]carbamoyl]-6-trifluoromethylindoline | | |
| SB 277011-A | N-{trans-4-[2-(6-cyano-3,4-dihydroisoquinolin-2(1H)- | 5.9 | pKᵢ |
| | yl)ethyl]cyclohexyl}quinoline-4-carboxamide | | |
| SDZ SER-082 | (+)-cis-4,5,7a,8,9,10,11,11a-octahydro-7H-10- | 6.7 | pKᵢ |
| | methylindolo[1,7-bc][2,6]-naphthyridine | | |
| spiperone | 8-[4-(4-fluorophenyl)-4-oxo-butyl]-1-phenyl-1,3,8- | 5.9-6.5 | pKᵢ |
| | triazaspiro[4.5]decan-4-one | | |
| | Espiperona | | |
| | Spiperonum | | |
| | Spiroperidol | | |
| | Spiropitan | | |
| spiroxatrine | 8-(7,10-dioxabicyclo[4.4.0]deca-1,3,5-trien-8-yl | 6.3-6.8 | pKᵢ |
| | methyl)-4-phenyl-2,4,8-triazaspiro[4.5]decan-1-one | | |
| | Espiroxatrina | | |
| | Spiroxatrinum | | |
| tegaserod | 1-[[5-(hydroxymethyl)-1H-indol-3-yl] | 8.4 | pKᵢ |
| | methylideneamino]-2-pentyl-guanidine | | |
| | HTF 919 | | |
| | SDZ HTF 919 | | |
| | Zelmac | | |
| terguride | N,N-diethyl-N′-[(8α)-6-methylergolin-8-yl]urea | 8.2 | pKᵢ |
| | dironyl | | |
| TIK-301 | 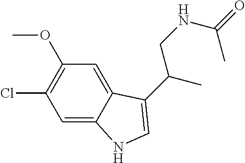 beta-methyl-6-chloromelatonin | | |
| trazodone | 8-[3-[4-(3-chlorophenyl)piperazin-1-yl]propyl]-6,8,9- | 7.1 | pKᵢ |
| | triazabicyclo[4.3.0]nona-2,4,9-trien-7-one | | |
| | Beneficat | | |
| | Bimaran | | |
| | Desirel | | |
| | Desyrel | | |
| | Molipaxin | | |
| | Trazalon | | |
| | Trazodil | | |
| | Trazodon | | |
| | Trazonil | | |
| xanomeline | 3-(4-hexoxy-1,2,5-thiadiazol-3-yl)-1-methyl-5,6- | 7.7 | pKᵢ |
| | dihydro-2H-pyridine | | |
| | LY 246708 | | |
| | Aphrosol | | |
| | Corynine | | |
| | Quebrachin | | |
| | Quebrachine | | |
| | Yohimbin | | |
| yohimbine | 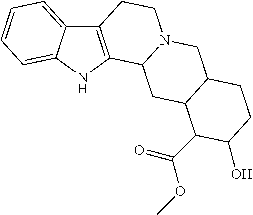 | 7.4-7.9 | pKᵢ |

In some embodiments, the ligand is preferably RS127445 or SB242084.

In yet a further embodiment, the invention provides a medicament comprising, separately or together:

(A) a compound of formula I or II, or a pharmaceutically acceptable salt thereof, and

(B) an anti-inflammatory agent,

for simultaneous, sequential or separate administration in the treatment of inflammation.

In yet a further embodiment, the invention provides a medicament comprising, separately or together:

(A) a compound of formula I or II, or a pharmaceutically acceptable salt thereof, and

(B) analgesic agent,

for simultaneous, sequential or separate administration in the treatment of pain.

In some embodiments of the invention, the anti-inflammatory agent defined in (B) is methotrexate.

In some embodiments of the invention, (B) is not a compound of formula I or II, or a pharmaceutically acceptable salt thereof.

In other embodiments of the invention, (A) or (B) is one of the 5HT_{2B} receptor antagonists defined above in Table 1.

In accordance with the above, the invention also provides a pharmaceutical kit comprising (A) and (B) as hereinabove defined in separate unit dosage forms, said forms being suitable for administration of (A) and (B) in effective amounts.

In another aspect, the present invention provides a pharmaceutical composition comprising a mixture of effective amounts of (A) as hereinabove defined and (B) as hereinabove defined, optionally together with at least one pharmaceutically acceptable carrier.

### BRIEF DESCRIPTION OF THE FIGURES

FIG. 1. N-(2-chloro-3,4-dimethoxybenzylideneamino)guanidine inhibited the binding of a receptor ligand to human 5-HT_{2B}, 5-HT_{2C} and 5-HT₄ receptors.

FIG. 2. Antagonistic effect of N-(2-chloro-3,4-dimethoxybenzylideneamino)guanidine on 5-HT_{2B} and 5-HT_{2C} receptors. The functionality of the compound was evaluated with the 5-HT_{2B} tissue assay and the 5-HT_{2C} GTPγS binding assay methods.

FIG. 3. Total RNA purified from different tissues or cells was reverse-transcribed and amplified for analysis of mRNA expression of the various 5-HT₂ receptor subtypes. Transcripts for 5-HT_{2B} and 5-HT_{2C} were detectable in rat synoviocytes and pannus while transcripts for 5-HT_{2A} were not detectable in the same RNA preparations.

FIG. 4. N-(2-chloro-3,4-dimethoxybenzylideneamino)guanidine (named CPD in figure) reversed the effects induced by BW723C86 and CP809101 (selective 5-HT_{2B} and 5-HT_{2C} receptor agonists, respectively).

FIG. 5. The effect of orally administered N-(2-chloro-3,4-dimethoxybenzylideneamino)guanidine (named CPD in figure) on the development of knee swelling in AIA (mean±SEM). The results of treatment with Methotrexate (MTX) are shown for comparison. Six animals were used in each group. Mann Whitney U-test gives at day 3: P<0.05 for 10 mg/kg and P<0.01 for 3 mg/kg.

FIG. 6. Effect of N-(2-chloro-3,4-dimethoxybenzylideneamino)guanidine (named CPD in figure) (1, 10 or 30 mg/kg) on inflammatory pain in rats. The compound was orally administered 60 min before formalin (vehicle) injection. The total time of the nociceptive response is presented for Phase 2 (15-60 min). N-(2-chloro-3,4-dimethoxybenzylidene-amino)guanidine showed a significant inhibition of nociceptive response on phase 2 compared to formalin at 30 mg/kg. Data are presented as mean±SEM. Significant differences were calculated using one-way ANOVA followed by Bonferroni's post test (**P<0.01 vs formalin), (n=10 animals/group).

FIG. 7. Rat synoviocytes were stimulated with 1 μM 5-HT in the presence of LPS (50 ng/ml) with the simultaneous addition of different concentrations (0.1, 1 or 10 μM) of RS 127445 (5-HT2B antagonist) or SB 242084 (5-HT2C antagonist). IL-6 levels in the medium were assayed after 72 hours. The addition of selective 5-HT2B and 5-HT2C receptor antagonists at different concentrations decreased the IL-6 production. Addition of N-(2-chloro-3,4-dimethoxybenzylideneamino)guanidine (COMP) shows similar dose dependent reduction in IL-6 levels (A). Rat synoviocytes were stimulated with 1 μM BW 723C86 (5-HT2B agonist) or 1 μM CP 809101 (5-HT2C agonist) in the presence of LPS (50 ng/ml) with the simultaneous addition of 5-HT2B and 5-HT2C receptor antagonists at three concentrations. IL-6 levels in the medium were assayed after 72 hours. The addition of selective 5-HT2B and 5-HT2C receptor antagonists at different concentrations decreased the IL-6 production. Addition of N-(2-chloro-3,4-dimethoxybenzylideneamino)guanidine shows similar dose dependent reduction in IL-6 levels. Statistical calculations were made using one-way ANOVA followed by Bonferroni's post test (***P<0.001, **P<0.01 and *P<0.05 vs relevant agonist).

The present invention is further defined in the following Examples, in which parts and percentages are by weight and degrees are Celsius, unless otherwise stated. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions. Thus, various modifications of the invention in addition to those shown and described herein will be apparent to those skilled in the art from the foregoing description. Such modifications are also intended to fall within the scope of the appended claims. The disclosure of each reference set forth herein is incorporated herein by reference in its entirety.

### EXAMPLES

### Example 1

### Preparation of N-(2-chloro-3,4-dimethoxybenzylideneamino)guanidine acetate

A solution of 2-chloro-3,4-dimethoxybenzaldehyde (1.0 g, 5 mmol), aminoguanidine bicarbonate (0.68 g, 5 mmol) and acetic acid (1 ml), in 15 ml of methanol is heated at reflux for 10 min. The reaction mixture is cooled down to 0° C. and the residue is filtered off. The filtrate is evaporated under vacuum and the product is crystallised from ethanol. Yield of the title compound is 1.1 g (70%), M.p. 198-200° C.

### Example 2

### Inhibition of Receptor-Ligand Binding

Determination of binding affinities for various 5-HT receptors was performed by radio-ligand binding assays. Briefly, tissue or cells expressing the receptor of interest were suspended in incubation buffer and radio-ligands and 10 μM N-(2-chloro-3,4-dimethoxybenzylidene-amino)guanidine were added. After incubation, separation of bound and free radio-ligand was done by multiple washings with buffer. Details for each assay are presented in Table 2.

The results were presented as the percent inhibition of specific binding. Mean values for each assay are presented in Table 3. The IC₅₀ values were determined by a non-linear least square regression analysis using MathIQ™ (ID Business Solution Ltd., UK). The inhibition constants (Kᵢ) were calculated using the equation of Cheng and Prusoff (Cheng Y, Prusoff W H, Biochem. Pharmacol. 22:3099-3108, 1973) using the observed IC₅₀ of the tested compound, the concentration of radioligand employed in the assay and the historical values for the K_{D} of the ligand.

TABLE 2

| Assay | Source | Ligand | Non-specific | Incubation buffer | Incubation | Reference |
|---|---|---|---|---|---|---|
| 5-HT_{1A} | Human rec. | [³H]-8-OH-DPAT 0.5 nM | 8-OH-DPAT 1 μM | | | |
| | HEK293 cells | | | | | |
| 5-HT_{1D} | Human cortex | [³H]-5-CT 2 nM | 5-CT maleate (1 μM) | | | |
| 5-HT_{2A} | Human cortex | [3H]-Ketanserin (2 nM) | Ketanserin (3 μM) | | | |
| 5-HT_{2B} | Human rec. | [³H] Lysergic acid | 5-HT (10 μM) | 50 mM Tris-HCl pH 7.4, 4 mM | 60 min @ 37° C. | Bonhaus |
| | CHO.K1 cells | diethylamide (LSD) 1.2 nM | | CaCl₂, 0.1% Ascorbic | | |
| | | | | acid | | |
| 5-HT_{2C} | Human rec. | [³H] Mesulergine 1 nM | Mianserin 1 μM | 50 mM Tris-HCl pH 7.4, | 60 min @ 25° C. | Wolf |
| | CHO.K1 cells | | | 0.1% Ascorbic acid, 10 μM | | |
| | | | | Pargyline | | |
| 5-HT₃ | Human rec. | [³H] GR65630 35 nM | MDL-72222 (1 μM) | | | |
| | HEK293 cells | | | | | |
| 5-HT₄ | Guinea pig, | [³H] GR113808 0.2 nM | 5-HT (30 μM) | | | |
| | striata | | | | | |
| 5-HT_{5A} | Human rec. | [³H] Lysergic acid | Methiothepin mesylate (1 μM) | | | |
| | HEK293 cells | diethylamide (LSD) 1 nM | | | | |
| 5-HT₆ | Human rec. | [³H] Lysergic acid | Methiothepin mesylate | | | |
| | HEK293 cells | diethylamide (LSD) 1.5 nM | (0.1 μM) | | | |
| 5-HT₇ | Human rec. | [³H] Lysergic acid | Methiothepin | | | |
| | CHO cells | diethylamide (LSD) 2.5 nM | | | | |

(References: Bonhaus DW, et al. (1995). Br J Pharmacol. 115: 622-628; Wolf WA and Schutz JS. (1997). J. Neurochem. 69: 1449-1458.)

TABLE 3
Inhibition of 5-HT receptor binding by 10 μM
N-(2-chloro-3,4-dimethoxybenzylideneamino)guanidine

| Receptor | % Inhibition |
|---|---|
| Serotonin, 5-HT_{1A} | 17 |
| Serotonin, 5-HT_{1D} | −5.7 |
| Serotonin, 5-HT_{2A} | 45 |
| Serotonin, 5-HT_{2B} | 107 |
| Serotonin, 5-HT_{2C} | 80 |
| Serotonin, 5-HT₃ | 3.3 |
| Serotonin, 5-HT₄ | 99 |
| Serotonin, 5-HT_{5A} | 54 |
| Serotonin, 5-HT₆ | 2.6 |
| Serotonin, 5-HT₇ | 53 |

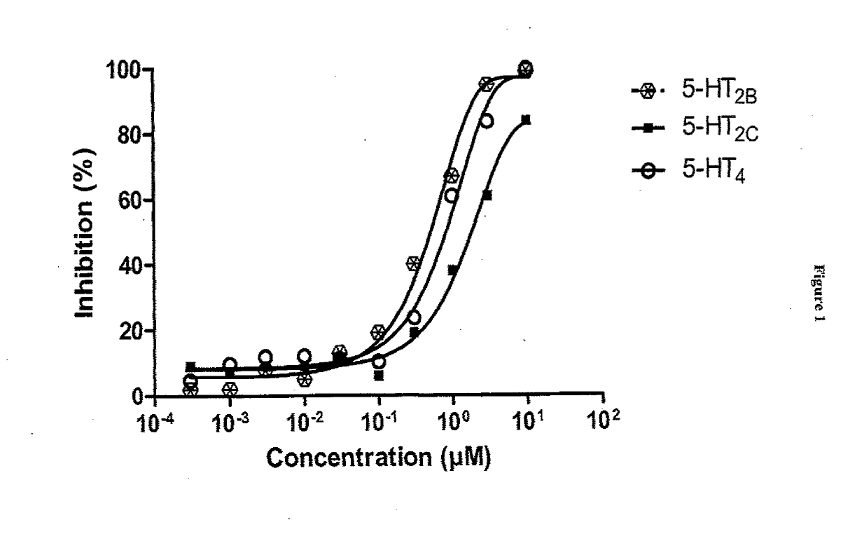

Ligand binding to three of the receptors was inhibited by more than 75% at 10 μM compound. These receptors, 5-HT_{2B}, 5-HT_{2C} and 5-HT₄, were further analysed at 10 different concentrations of the compound, resulting in Kᵢ values of 263, 880 and 276 nM, respectively (FIG. 1).

N-(2-chloro-3,4-dimethoxybenzylideneamino)guanidine was further tested for functionality against 5-HT_{2B} (Cohen et al. (1987), J. Pharmacol. Exp. Ther. 243:264-269), 5-HT_{2C} (Adlersber M, et al. (2000), J. Neurosci. Res. 61(6):674-685; Cussac D. et al. (2002), Mol. Pharmacol. 62(3):578-589) and 5-HT₄ (Reeves et al. (1991), Br. J. Pharmacol. 103:1067-1072).

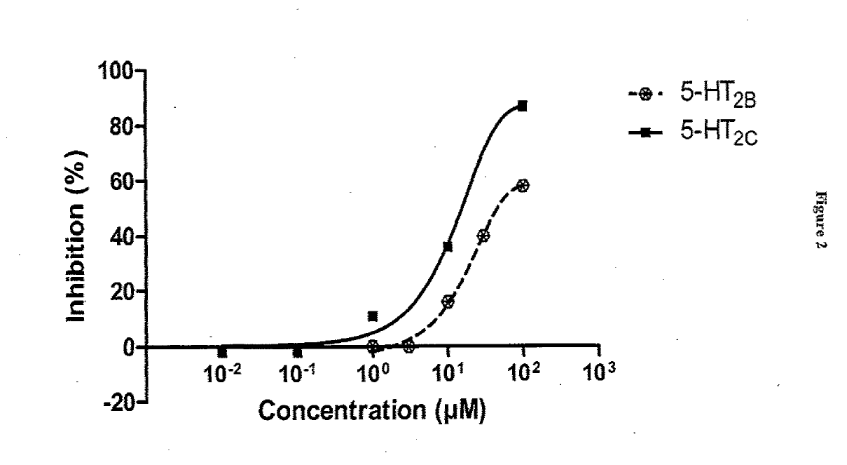

The compound has an antagonistic effect on 5-HT_{2B} and 5-HT_{2C} with IC₅₀ values of 61.7 and 16.5 μM, respectively (FIG. 2). There was no significant effect on 5-HT₄ at 100 μM.

### Example 3

### Antagonism of 5HT₂ Receptors

The 5-HT₂ receptor expression pattern on pannus and synovial cells was evaluated by RT-PCR. N-(2-chloro-3,4-dimethoxybenzylideneamino)guanidine was added in vitro to rat synoviocytes together with commercial 5-HT₂ receptor agonists to investigate their effects on IL-6 release.

### RNA Preparation

Total RNA was extracted from cell cultures or tissues using Qiashredder and RNeasy Mini Kit (Qiagen, Valencia, Calif., USA). Purified RNA was quantified by visualization at a wavelength of 260 nm (Jenway, 6405).

### Reverse-Transcriptase of Erase Chain Reaction (RT-PCR) Analysis of 5-HT₂ Receptor Expression

The primer sequences and their conditions for use are summarized in Table 4. 400 ng total RNA of each sample was reversely transcribed into cDNA in a 50 μl reaction using the Illustra™ Ready-to-Go RT-PCR Beads (GE-Healthcare, UK). One micro litre aliquots were amplified according to described reaction mixture. The following reaction profile was used for all experimental setup (experiments): a first strand cDNA synthesis reaction at 42° C. for 15 min, an initial denaturation at 94° C. for 2 min, 2 cycles at 94° C. for 15 sec; 50° C. for 15 sec; and 72° C. for 30 sec, 2 cycles at 94° C. for 15 sec; 52° C. for 15 sec; and 72° C. for 30 sec, 2 cycles at 94° C. for 15 sec; 54° C. for 15 sec; and 72° C. for 30 sec, 2 cycles at 94° C. for 15 sec; 56° C. for 15 sec; and 72° C. for 30 sec, 2 cycles at 94° C. for 15 sec; 58° C. for 15 sec; and 72° C. for 30 sec, followed by 35 cycles at 94° C. for 15 sec; 55° C. for 15 sec; and 72° C. for 30 sec, and an additional 1 min extension step at 72° C. after last cycle. Amplification reactions were performed in an Eppendorf Mastercycler ep (Eppendorf A G, Hamburg, Germany). A negative control, where reverse transcriptase was omitted from the reaction, was run in parallel and yielded no PCR-product. As a positive control total RNA isolated from rat brain was used. Internal positive controls consisted of GAPDH.

TABLE 4

| | | Size of | Annealing |
|---|---|---|---|
| Gene | Sequence 5′-3′ | product (bp) | (° C.) |
| 5-HT_{2A} rat | AGAACGCTCCGAACCCTAAT | 246 | 55 |
| (forw) | (SEQ ID NO: 1) | | |
| 5-HT_{2A} rat (rev) | AGCCAATCCACACAAACACA | | |
| | (SEQ ID NO: 2) | | |
| 5-HT_{2B} rat | GTCCTGCCTGGTTATTCCTTGATG | | |
| (forw) | (SEQ ID NO: 3) | 221 | 55 |
| 5-HT_{2B} rat (rev) | CGTTGACCACATCAGCCTCTATTC | | |
| | (SEQ ID NO: 4) | | |
| 5-HT_{2C} rat | AGCTCTGTGCGATCTGGATT | | |
| (forw) | (SEQ ID NO: 5) | 239 | 55 |
| 5-HT_{2C} rat (rev) | CCCCTCCTTAAAGACCTTCG | | |
| | (SEQ ID NO: 6) | | |
| GAPDH rat | CAACTCCCTCAAGATTGTCAGCAA | | |
| (forw) | (SEQ ID NO: 7) | ~150 | 55 |
| GAPDH rat (rev) | GGCATGGACTGTGGTCATGA | | |
| | (SEQ ID NO: 8) | | |

### Isolation, Culture and Stimulation of Rat Synoviocytes

Pannus from the inflamed knee of rats with antigen-induced arthritis (AIA) (see description of this model below; Andersson, S. E et al., (1998), J. Rheumatology 25: 1772-1777) was isolated three to five days after challenge, stored in PBS supplemented with 100 U/ml Penicillin-Streptomycin (PEST, Invitrogen Corporation) and 2.5 μg/ml Fungizone (Amphotericin B, Invitrogen) until it was further processed within a couple of hours. The PBS was discarded and the tissue cut in small pieces followed by digestion in a collagenase solution (400 U/ml; type 1 CLS-1, Worthington) for 3 hours at 37° C., 5% CO₂. The suspension was filtered through a nylon mesh (70 μm), washed with tissue culture medium (RPMI-1640 with L-glutamine, Invitrogen, supplemented with 10% fetal bovine serum (FBS) and 100 U/ml PEST and 2.5 μg/ml Fungizone) and centrifuged (257 g). The cells were resuspended in tissue culture medium and seeded in cell culture flasks (T25, TPP). After overnight culture, nonadherent cells were removed by exchanging the medium, the cells were incubated at 37° C., 5% CO₂, the medium was exchanged every third day until confluence was reached (7-10 days). The culture consisted of a mixed cell population with the principal constituents being fibroblasts and macrophages (Andersson, S. E. et al. (2000), Eur. J. Pharm. Sci 9: 333-343).

The cells were counted and reseeded at a density of 10000 cells/0.2 ml in 96-well cell culture plates (Nunc). After overnight culture the cells were stimulated with 50 ng/ml Lipopolysaccharide (LPS, E. coli 055:B5, Sigma) with simultaneous addition of 0.1, 1 and 10 μM N-(2-chloro-3,4-dimethoxybenzylideneamino)guanidine and 1 μM of 5-HT₂ receptor agonists (triplicates). The cells were incubated for 72 hours after which the supernatants were collected and analyzed for IL-6 (OptEIA™ Set Rat IL-6). The effect of treatment on cell viability was determined using the cell proliferation reagent, WST-1 (Roche Diagnostics).

### Results

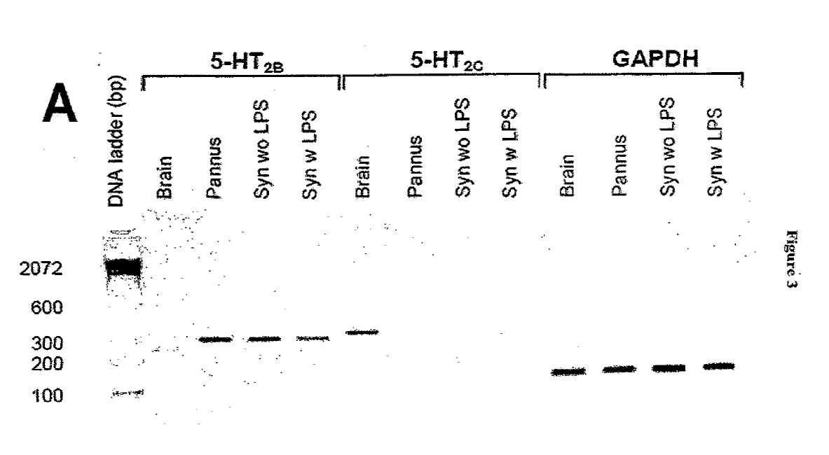

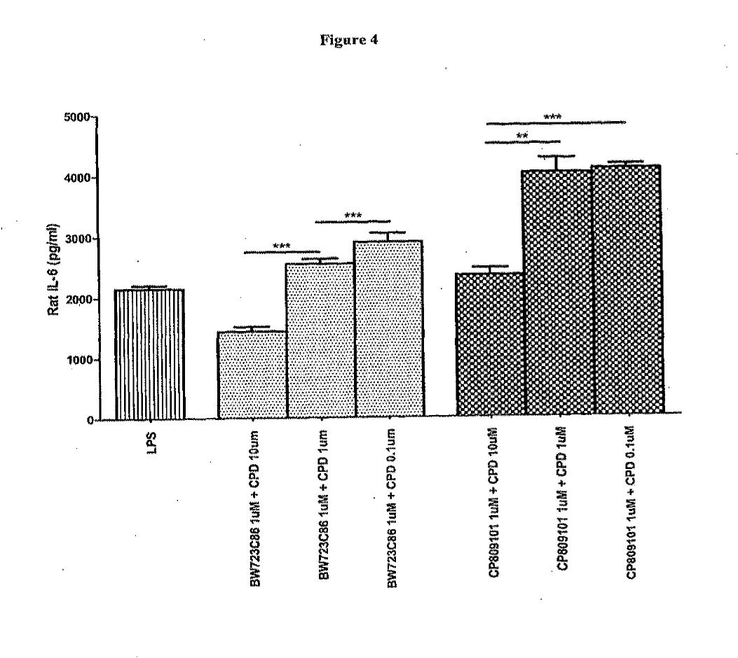

In vitro target validation experiments showed that the 5-HT₂ receptors are expressed on the target cells (FIG. 3) and that N-(2-chloro-3,4-dimethoxybenzylideneamino)guanidine reversed the effects induced by selective 5-HT₂ receptor agonists (FIG. 4) and thus acts through the 5-HT₂ receptors.

### Example 4

### Effects on Inflammation in Antigen-Induced Arthritis in the Rat

Antigen-Induced Arthritis (AIA) is a Well-Documented Animal Experimental Model of human arthritic disease (Dumonde, D. C. and Glynn, L. E. (1962), Br. J. Exp. Pathol. 43:373-383). In this model, inflammation is induced by immunization followed by an intra-articular (knee-joint) challenge with the antigen. This causes an increase in knee diameter due to the formation of a pannus, i.e. hyperproliferative synovial tissue, which spreads over the articular cartilage into the bone, leading to erosion and destruction of the joint tissue (Carpenter, T. A. et al. (1994), Skeletal Radiol. 23:429-437).

AIA was applied for a short-term in vivo evaluation of our test compounds, with treatment during 4 days. The test compounds were compared with methotrexate, a well-documented “gold standard” drug for the treatment of rheumatoid arthritis (Bannwarth, B. et al. (1994), Drugs 47:25-50), which has been shown to have an anti-arthritic effect in this model (Andersson, S. E. et al. (2000), Eur. J. Pharm. Sci. 9: 333-343).

### Experiments

Female Dark Agouti rats were sensitized subcutaneously at the tail root with 1 mg mBSA (Sigma A1009) dissolved in 50 μl saline and emulsified in 50 μl Freund's complete adjuvant (Sigma F5881). Eleven days later the rats were challenged (unilateral) with an intra-articular (i.a.) injection of 75 μg mBSA dissolved in 50 μl saline. The procedures were carried out under a brief Isoflurane anaesthesia. Animals were treated once or twice daily with oral administration of N-(2-chloro-3,4-dimethoxybenzylideneamino)guanidine in saline. The treatment was started on the day of challenge, and was continued for 4 days. The knee-joint swelling was measured daily with an odimeter/calliper; the body weight was recorded before arthritis induction and at the end of the experiment. The test compound was administered orally, at 1 to 10 mg/kg body weight. Methotrexate was given once, 2.5 mg/kg.

### Results

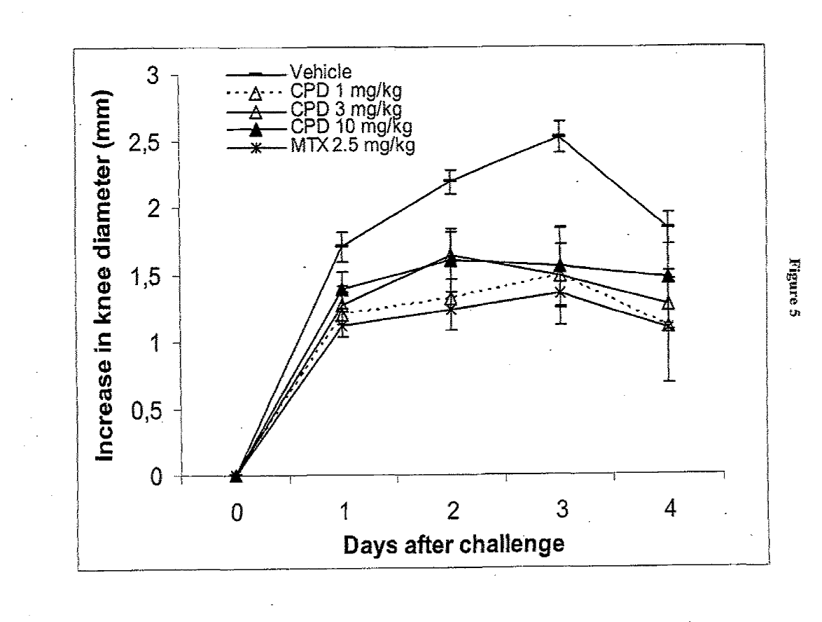

The compound was found to reproducibly reduce joint swelling in the dose range of 1-10 mg/kg (FIG. 5). The beneficial effect of the compound, administered orally twice daily at 10 mg/kg, was reproduced by administration of the same dose once daily. In addition, a therapeutic effect of the compound was found at administration one day after disease induction.

### Example 5

### Inflammatory Pain Model

Inflammatory pain is associated with tissue damage and the resulting inflammatory process is related to conditions such as arthritis and many autoimmune disease states. The formalin test in rats is a widely used tool when studying inflammatory pain in animals (Dubuisson, D., and Dennis, S. G. (1977), Pain 4:161-174; Wheeler-Aceto, H., et al. (1990), Pain 40:229-238). This model allows the study of two different types of pain that appears in two separate phases: phase 1 which is a direct stimulation of the nerve by the formalin and thus resembles the acute pain, followed by phase 2 which is an inflammatory-reaction induced pain similar to the pain in, e.g., arthritis.

### Experiments

Animals (Male Sprague Dawley rats provided by Charles River, Calco, Lecco, Italy) were acclimatized for 60 minutes in single Plexiglas cage to the testing environment before assessment. 50 μl of 5% of formalin was s.c. administered into the dorsum of the right hind paw, using micro-syringe with a 29-gauge needle. After formalin injection the rats were immediately released in the testing cage and their behaviours were observed. Food and water availability had been refused starting ten and two hours respectively before testing and for the experimental course.

In accordance with the literature (Sufka K J, et al., Eur J Pain (1998), 2(4):351-8; Maione S, et al., Br. J. Pharmacol. 2007; 150:766-781), nociceptive response was monitored in consecutive 5-min periods for 60 min following formalin injection. In particular, nociceptive responses were recorded by measuring the cumulative time of lifting, licking, shaking and flinching of the injected paw per unit time (5 minutes). The nociceptive response was measured both as every 5 minutes and as total time of the first phase (0-15 min) and the second phase (15-60 min). The nociceptive response was expressed in minutes (mean±SEM). N-(2-chloro-3,4-dimethoxybenzylideneamino)guanidine (1, 10 and 30 mg/kg) was orally administered to rats 60 minutes before formalin injection to determine dose-response prevention of the formalin-induced pain.

### Results

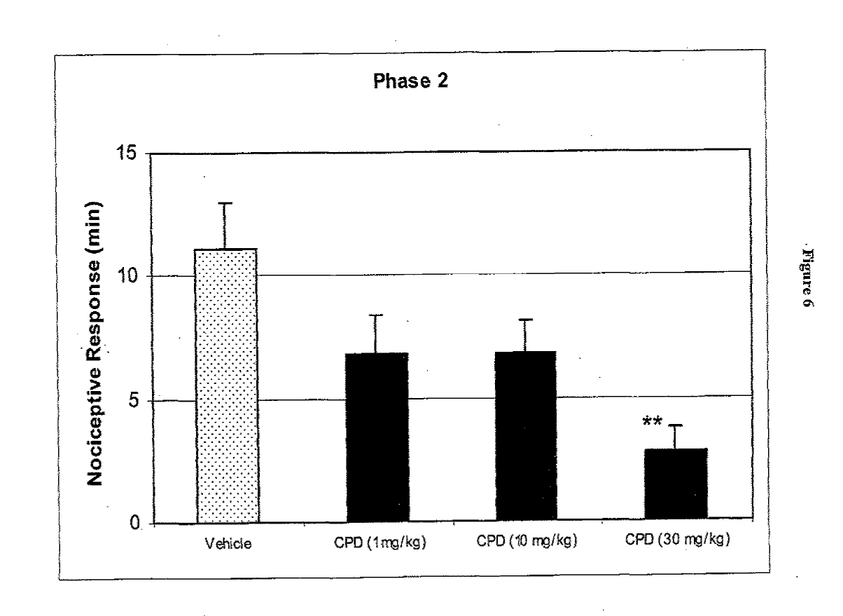

N-(2-chloro-3,4-dimethoxybenzylideneamino)guanidine significantly reduced the inflammatory pain response at 30 mg/kg (FIG. 6), without significantly influencing the first part of the nociceptive response.

### Example 6

### Modulation of Inflammation via the 5-HT2B and 5-HT2C Receptors

To evaluate the effect of 5-HT stimulation on the inflammatory response in rat synoviocytes, changes in IL-6 expression were measured. Also, the effect of including commercial 5-HT2 receptor antagonists was investigated.

Inflamed knee joints of rats with antigen-induced arthritis (AIA) were dissected four days after intra-articular challenge with antigen. Freshly isolated pannus tissue was collected in phosphate-buffered saline (PBS) supplemented with 100 U/ml penicillin, streptomycin (PEST, Invitrogen) and 2.5 μg/ml Fungizone (Amphotericin B, Invitrogen), finely minced with a pair of scissors and digested with 400 U/ml collagenase (type 1 CLS-1, Worthington) in RPMI 1640 (Gibco) supplemented with PEST and fungizone (2.5 μg/ml) for 3 hours. The cell suspension was filtered through a 70 μm nylon mesh and the cells collected by centrifugation at 257×g, 10 min. The cells were resuspended in RPMI 1640 supplemented with 10% FBS (Gibco), PEST and fungizone (culture medium), plated in T25 flasks (TPP Cat#90026). After overnight incubation nonadherent cells were removed and the culture medium was replaced every third day until the cells approached confluency (normally achieved within 7-10 days). At near-confluency, the cells were washed with PBS and harvested by trypsination. After washing in PBS the cells were resuspended in culture medium and seeded at 10.000 cells/well/0.2 ml in 96 well plates (Nunc cat#167008). After overnight culture the culture medium was replaced with fresh medium and 50 ng/ml LPS (Sigma, L6529) together with different concentrations of 5-HT, 5-HT2 receptor agonists and/or antagonists and N-(2-chloro-3,4-dimethoxybenzylideneamino)guanidine. The cells were incubated for 72 hours after which the supernatants were collected and analyzed for IL-6 (BD OptEIA™ Set). The effect of treatment on cell viability was determined using the cell proliferation reagent, WST-1 (Roche Diagnostics). In another experiment rat synoviocytes were incubated with 50 ng/ml LPS for 2 or 22 hours were after the levels of 5-HT was analysed in the cell culture medium using a Serotonin ELISA kit (IBL International, RE59121). We also incubated rat synoviocytes with 5-HT (10 μM) and measured the gene expression of 5-HT2B and 5-HT2C.

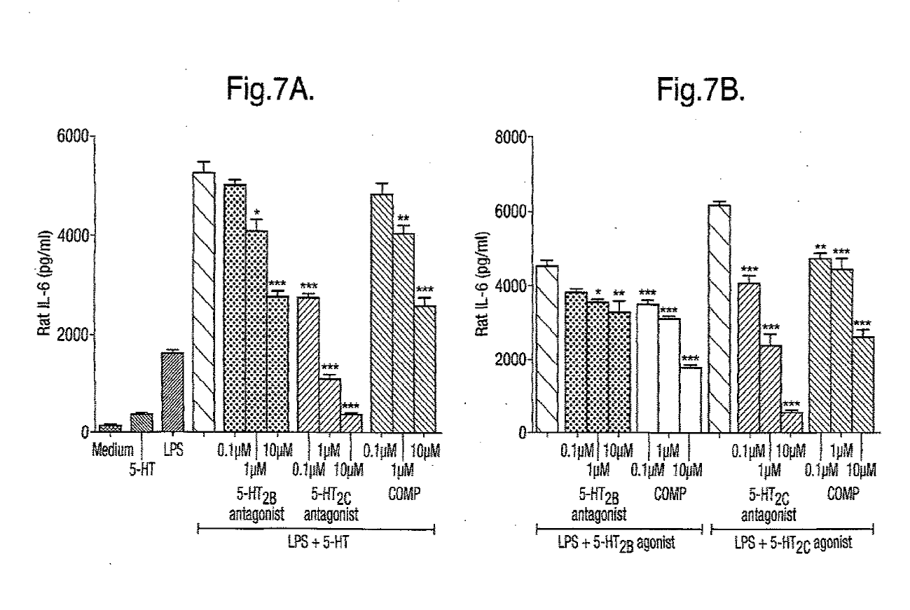

FIG. 7A shows a clear increase in IL-6 release when 5-HT was added, in line with the described pro-inflammatory role of 5-HT. A decrease in IL-6 release could subsequently be obtained by the addition of partly selective 5-HT2 receptor antagonists. The addition of different concentrations of 5-HT2B (RS127445) and 5-HT2C(SB242084) receptor antagonists respectively, clearly reduced the IL-6 release. A reduction in IL-6 levels was also observed when N-(2-chloro-3,4-dimethoxybenzylideneamino)guanidine was included, indicating an anti-inflammatory effect mediated through the antagonistic binding to the 5-HT2B and 5-HT2C receptors. In conclusion these results show that 5-HT is capable of inducing an inflammatory response in rat synoviocytes and that compounds with a described antagonistic effect on the respective receptor subtype, 5-HT2B and 5-HT2C reduce the inflammatory response measured as changes in IL-6 release. In FIG. 7A we also show that the addition of 10 μM N-(2-chloro-3,4-dimethoxybenzylideneamino)guanidine reduces the IL-6 response induced by 1 μM 5-HT and LPS. These results support the hypothesis that 5-HT induces an inflammatory response that could be counteracted by N-(2-chloro-3,4-dimethoxybenzylideneamino)guanidine.

To further discriminate between the different 5-HT2 receptors we used selective commercial 5-HT2 receptor agonists to stimulate rat synoviocytes. The agonists were separately given to synoviocytes to enhance IL-6 release. N-(2-chloro-3,4-dimethoxybenzylideneamino)guanidine was simultaneously added to the cells in order to evaluate which of the agonists caused a response that could be counteracted by our compound. FIG. 7B clearly demonstrates that the 5-HT2B receptor agonist, BW 723C86, induces a response in IL-6 production that in a dose-dependent way is counteracted by N-(2-chloro-3,4-dimethoxybenzylideneamino)guanidine. In a similar way CP 809101, 5-HT2C receptor agonist, is counteracted by N-(2-chloro-3,4-dimethoxybenzylideneamino)guanidine. In combination with results from the receptor expression analysis we could conclude that the decrease in IL-6 release achieved with N-(2-chloro-3,4-dimethoxybenzylideneamino)guanidine, is mediated by the 5-HT system and that both the 5-HT2B and 5-HT2C receptor most likely are involved.

1-21. (canceled)

22. A method of treating rheumatoid arthritis or osteoarthritis or pain comprising administering an effective amount of a compound of formula (I) 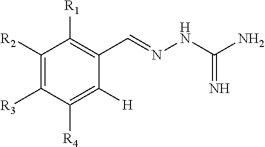
wherein
R1 is Cl, MeO or H
R2 is Cl, MeO or H
R3 is Cl or MeO
R4 is Cl, H or NO₂
with the proviso at least one of R1-R3 must be Cl
and at least one of R1-R3 must be MeO,
or a pharmaceutically acceptable salt thereof,
to a patient in need thereof.

23. A method as claimed in claim 22, wherein R1 is Cl.

24. A method as claimed in claim 22, wherein R2 is MeO.

25. A method as claimed in claim 22, wherein R3 is MeO.

26. A method as claimed in claim 22, wherein R4 is H.

27. A method as claimed in claim 22, comprising administering a compound of formula II: 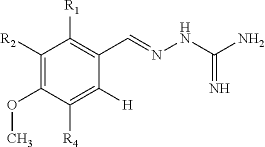
wherein
R1 is Cl or MeO, preferably Cl,
R2 is Cl or MeO, preferably MeO,
R4 is Cl or H, preferably H,
with the proviso that at least one of R1 and R2 must be Cl,
or a pharmaceutically acceptable salt thereof.

28. A method as claimed in claim 22, wherein the compound is N-(2-chloro-3,4-dimethoxybenzylideneamino)guanidine or a pharmaceutically acceptable salt thereof.

29. A method as claimed in claim 22, wherein the pain is nociceptive pain.

30. A method as claimed in claim 28, wherein the pain is nociceptive pain.


SEQUENCE LISTING (8 sequences)

SEQ ID NO: 1 - DNA, 20 bp, Rattus sp.
agaacgctccgaaccctaat

SEQ ID NO: 2 - DNA, 20 bp, rattus sp.
agccaatccacacaaacaca

SEQ ID NO: 3 - DNA, 24 bp, rattus sp.
gtcctgcctggttattccttgatg

SEQ ID NO: 4 - DNA, 24 bp, rattus sp.
cgttgaccacatcagcctctattc

SEQ ID NO: 5 - DNA, 20 bp, rattus sp.
agctctgtgcgatctggatt

SEQ ID NO: 6 - DNA, 20 bp, rattus sp.
cccctccttaaagaccttcg

SEQ ID NO: 7 - DNA, 24 bp, rattus sp.
caactccctcaagattgtcagcaa

SEQ ID NO: 8 - DNA, 20 bp, rattus sp.
ggcatggactgtggtcatga